(19) United States
(12) Patent Application Publication
(10) Pub. No.: US 2008/0226492 A1
(43) Pub. Date: Sep. 18, 2008

(54) Deactivating Emulsions

(75) Inventors: Sabrina Higgins, Southampton (GB); John Hughes, Southampton (GB); Malcolm Tom Mckechnie, Hull (GB)

Correspondence Address: NORRIS, MCLAUGHLIN & MARCUS, 875 THIRD AVE, 18TH FLOOR, NEW YORK, NY, 10022, US

(73) Assignees: RECKITT BENCKISER (UK) LIMITED, Slough, Berkshire (GB); THE UNIVERSITY OF SOUTHAMPTON, Highfield, Southampton (GB)

(21) Appl. No.: 10/597,463

(22) PCT Filed: Feb. 14, 2005
(86) PCT No.: PCT/GB2005/000502

§ 371 Date: Sep. 6, 2006

(30) Foreign Application Priority Data
Feb. 13, 2004 (GB) 0403232.2

(51) Int. Cl. A61L 9/02

(52) U.S. Cl. 422/4; 516/53

(57) ABSTRACT

A method of deactivating an allergen involves dispersing into an airspace an allergen deactivant, the deactivant being provided in the form of an oil-in-water emulsion comprising at least 8 wt % deactivant. Preferably the deactivant is dispersed into the airspace as a vapour. Preferably the dispersal is aided by heat applied to the emulsion.

The present invention relates to a method of deactivating dust mite allergens.

Various allergens are known to trigger a human reaction. For example, it has been known for a long time that house dust can trigger allergenic reactions in humans, such as asthma and rhinitis. It was reported, as early as 1928 that it was the dust mites in the dust that were the primary source of the allergenic response, but it was only in the 1960's that researchers appreciated its significance.

House dust mites produce detritus which causes allergenic reaction in many people. The major allergens are believed to be detritus from the mite species Dermatophogoides farinae and Dermatophagoides pteronyssinus (the allergens being known as Der f1 and Der p1 respectively). The detritus includes faeces as well as body part residues of the mites. A review is given in Experimental and Applied Acarology, 10 (1991) p. 167-186.

Other allergens which are problematic include cockroach allergens (notably the Bla g1 cockroach allergen), and cat allergens (Fel d1). In the case of cat allergens the coat/fur of the cat and/or its salivary deposits seem to be of significance in eliciting the allergenic response.

WO99/15208 describes a method for deactivating allergens derived from the D. Pteronyssinus and D. Farinae dust mite species, which comprises contacting the allergen with one of 28 deactivants which are described. These may be delivered into an airspace by aerosol spraying.

WO 01/76371 describes further deactivants for house dust mite allergens. These may be delivered into an airspace by various methods including by use of heat to vaporise an oil, an ultra-sonic jet nebuliser, an ion wind, or a candle incorporating a deactivant. In the case of the oil, this may be used as such or may be floated on water or may be presented in the form of an oil-in-water emulsion, generally having up to 5% by weight of the oil.

In accordance with a first aspect of the present invention there is provided method a of deactivating an allergen, the method comprising dispersing into an airspace an allergen-deactivating amount of an allergen-deactivating compound (hereinafter the “deactivant”), the deactivant being provided in the form of an oil-in-water emulsion comprising at least 8% of a deactivant (wt. deactivant/wt. emulsion), and being dispersed into the airspace as a vapour.

Preferably the emulsion comprises at least 9%, and most preferably at least 10%, of a deactivant.

Preferably the deactivant used in the method of the present invention is provided in the form of an oil-in-water emulsion comprising up to 25% of deactivant, preferably up to 20%, more preferably up to 18% and most preferably up to 15% of deactivant.

An especially preferred oil-in-water emulsion for use in the method of the present invention comprises 12% of a deactivant.

The percentages given in the above definitions denote the total deactivant content, when there is more than one deactivant present.

In this specification unless otherwise stated a percentage value given for a component denotes the weight of the component expressed as a percentage of the total weight of the emulsion.

Use of the noun deactivant and the verb deactivate in this specification denote that some or all of a source of allergens at a locus are rendered unable to evoke an allergenic response in a human, by a method of the present invention. The net result is that the source may be reduced in its allergenicity, or its allergenicity may be completely removed.

Preferably the deactivant is selected from:
a terpene hydrocarbon;
a citrus oil;
a mint oil;
bois de rose oil;
oil of jasmine;
frankincense;
oil of bergamot; and
oil of lemon grass.

Preferred terpene hydrocarbons include tea tree oil, pinol and β-pinene.

An especially preferred deactivant is a citrus oil, most preferably orange oil.

Another especially preferred deactivant is β-pinene.

A deactivant may suitably be a single compound. Alternatively a mixture of deactivants may be used together.

A deactivant may be part of a blend of compounds, not all of which are deactivants. For example a citrus oil is a blend of compounds not all of which will function as deactivants.

A deactivant may suitably be dispersed into the airspace over an extended period, for example at least 30 minutes, and preferably at least 1 hour.

A deactivant may suitably be dispersed into the airspace on two occasions, interrupted by a period in which there is no deactivant dispersal. A deactivant may be dispersed into the airspace on one or more further occasions, following a corresponding period or periods of no deactivant dispersal. Preferably each such dispersal occasion involves deactivant dispersal over an extended period, as described above. Preferably the or each period in which there is no deactivant dispersal is an extended period, for example at least 2 hours, preferably at least 4 hours, and most preferably at least 8 hours.

We have found that the method produces a prolonged reduction in the allergen loading of an allergen-contaminated inanimate substrate. Delivery of the deactivant into an airspace as described causes a permanent reduction in the population of allergens in an inanimate test source. By inanimate test source we mean a test source which is itself inanimate (e.g. it is not the skin or coat/fur of a live animal) and it does not contain living organisms, such as dust mites. Populations of dust mites would make any result difficult to interpret.

We have found that the reduction in allergen content in such a source is of long duration, for example at least 7 days, typically at least 14 days, and suitably at least 28 days. Indeed, in tests we have carried out over a 28-day period, we have found that the allergen content may continue to decline over time, even though the deactivant may have been used days or weeks before. The results suggest that the allergenic species have been truly denatured or degraded, to the extent that, firstly, they cannot re-form, and secondly, their degradation products are not themselves allergenic. It further suggests that the action of the deactivant is not merely a masking or damping effect. Any such effect would be likely to break down over time.

The formation of emulsions is generally well known in the art and is described, for example, in Modern Aspects of Emulsion Science, edited by Bernard P. Binks, The Royal Society of Chemistry, 1998 and Surfactant Science and Technology, Second Edition, Drew Myers, 1992, VCH Publishers, Inc. Non-ionic surfactants may be especially suitable. Proprietary surfactant packs may be employed to form emulsions, for example E-Z-MULSE (Trade Mark), a non-ionic surfactant pack from Florida Chemical Company, US.

Preferably the deactivant is dispersed into the airspace as a vapour.

Preferably the dispersal of the deactivant is aided by heat applied to the emulsion.

A heat source is preferably located beneath a source of the emulsion. This may, for example, be an oil burner, candle or an electrical heat source, such as a hot plate. Preferably it is a hot plate, preferably having a temperature of at least 100° C.

The use of a hotplate enables the heat applied to vaporise the deactivant to be controlled, in a manner which is not possible with prior methods.

Our work suggests that use of a hotplate below 100° C. gives some allergen deactivating activity but that use of a higher temperature gives allergen deactivating activity of a substantially and surprisingly higher level, even though the quantity of deactivant dispersed may be the same in each case.

Preferably the hotplate has an electrical heat source.

Preferably the vessel and the hotplate are in face-to-face contact. Preferably the hotplate has a flat surface and the vessel has a flat base, and the vessel rests on the hotplate. Preferably the vessel has an opening in its upper region. Preferably it has a fully open upper face. Preferably, therefore, the vessel has a flat base, a side (if cylindrical) or sides depending upwardly therefrom, and no further side.

Preferably the hotplate is at a temperature of at least 130° C.

Preferably the hotplate is at a temperature up to 300° C., preferably up to 250° C.

The present invention involves the dispersal of an allergen deactivant into an airspace. It is possible that airborne allergens may be deactivated but it is believed that there is effective deactivation of allergens borne on surfaces within the airspace.

In accordance with a further aspect of the present invention there is provided the use of an oil-in-water emulsion in deactivating an allergen at a locus, the emulsion comprising 10-15% of an allergen deactivant, a heat source being used to accelerate the vaporization of the deactivant.

In accordance with a further aspect there is provided an allergen-deactivating oil-in-water emulsion comprising at least 8% of a volatile deactivant, wherein the deactivant is selected from:
a terpene hydrocarbon;
a citrus oil;
a mint oil;
bois de rose oil;
oil of jasmine;
frankincense;
oil of bergamot; and
oil of lemon grass.

Preferably an allergen deactivated in a method or use in accordance with the present invention is a material which evokes an allergenic reaction in a human. For example it may be an allergen arising from house dust mites, or from pets. Most preferably the method or use of this invention is able to deactivate, partially or wholly, an allergen arising from the mite species Dermatophogoides farinae (known as Der f1) or, especially from the mite species Dermatophagoides pteronyssinus (known as Der p1). Cat allergens (Fel d1) and cockroach allergens (Bla g1) may also be deactivated.

The present invention will be further described with reference to the following Examples.

### EXPERIMENTAL PROTOCOL

When using house dust for allergen denaturing tests an inherent difficulty is the variability of the amount of allergen in each small sample, even when taken from the same dust reservoir. The amount of dust in the pre-treatment sample must be accurately estimated in order to determine the extent of any allergen denaturing. In these tests the dust sample was applied to the test exposure surface and then one half of this surface dust was removed to measure the control pre-treatment allergen level of that specific sample. Each control was directly relevant to each sample, which gave the best possible estimate of the level of allergen in the sample before exposure to possible denaturant. All tests employed a glass reinforced plastic booth of size 0.7 m×0.7 m×1.0 m. Average values are stated.

The following Examples all measure the reduction of the allergen Der p1 from the house dust mite Dermatophagoides pteronyssinus.

House dust was passed through a number of sieves and the fraction smaller than 53 μm was collected. 0.025 g of dust was placed in a small sieve to distribute it evenly over the test surface. The test surface was a PTFE (polytetrafluoroethylene—registered trade mark TEFLON) coated metal tray of size 30 cm×30 cm. The dust was applied to the tray by moving the sieve continuously over the surface while tapping the sieve. One half of the dust was then removed by suction onto an in-line filter and the weight recorded, this was the pre-treatment control.

The tray was then placed in the booth. Three tea light holders—upwardly open cylindrical vessels (diameter 40 mm, height 15 mm) produced to hold nightlight candles—each containing 6 ml of water and 0.8 ml of orange oil—were placed together on an electric hotplate set to 250° C. In practice we found that this meant that the hotplate temperature cycled between 130° C. and 250° C. The booth was sealed. Heat was delivered for the period specified below, and then the hotplate was allowed to cool. After 24 hours the tray was removed, the dust was collected from it and its weight recorded. The booth was washed with strong detergent between tests.

Identical tests were carried out differing only in their test liquids. These were:
5% orange oil floated on water (evaporated in 29 minutes)—comparative
12% orange oil floated on water (evaporated in 30 minutes)—comparative
20% orange oil floated on water (evaporated in 20 minutes)—comparative
50% orange oil floated on water (evaporated in 20 minutes)—comparative
12% orange oil/water emulsified with E-Z-MULSE—of the invention (heating stopped after 105 minutes; did not evaporate to dryness. This is believed to be due to remaining non-volatile surfactant from E-Z-MULSE constituent).

The test samples were assayed for the Der p1 allergen using an ELISA (Enzyme linked immunosorbent assay) to determine the allergen content. This was then related to the weight of dust that had been present in each sample. All of the samples were multiplied up to compare the amount of allergen expected to be present in a 0.1 g sample of dust. The percentage difference between the control sample and the exposed sample was then obtained.

The Der p1 allergen reductions were as follows:
5% orange oil-on-water—11.9%
12% orange oil-on-water—75.4%
20% orange oil-on-water—67.0%
50% orange oil-on-water—68.1%
12% orange oil emulsion—91.0%

The non-volatility of the surfactant content of the emulsion suggested that the orange oil, in emulsion form, was responsible for the activity increase, not the surfactant content itself.

It was found that the allergen content did not substantially recover over time.

Statistical analysis suggested that the increase in activity, from the 12% oil-on-water test liquid to the 12% emulsion, was a significant result.

1. A method of deactivating an allergen from the mite species Der f1 or Der p1, the method comprising the step of:
dispersing into an airspace an allergen-deactivating amount of an allergen-deactivating compound, said compound being provided in the form of an oil-in-water emulsion comprising at least 8% weight of a deactivant, said emulsion being dispersed into the airspace as a vapour.

2. A method as claimed in claim 1, wherein the allergen-deactivating compound is dispersed into the airspace over a period of at least 30 minutes.

3. A method according to claim 1, wherein the dispersal is aided by heat applied to the emulsion.

4. A method according to claim 1 wherein the allergen-deactivating compound is selected from:
a terpene hydrocarbon;
a citrus oil;
a mint oil;
bois de rose oil;
oil of jasmine;
frankincense;
oil of bergamot;
oil of lemon grass;
or a component thereof.

5. A method according to claim 1 wherein the allergen-deactivating compound comprises a terpene hydrocarbon.

6. A method according to claim 1 wherein the allergen-deactivating compound comprises β-pinene.

7. A method according to claim 1 wherein the allergen-deactivating compound comprises orange oil or a component thereof.

8. A method of deactivating an allergen at a locus, the method comprising the step of providing an oil-in-water emulsion an allergen deactivant present in a concentration of 10-15% wt./wt. of emulsion, and heating the said emulsion with a heat source to accelerate the vaporization of the deactivant.

9. An allergen-deactivating oil-in-water emulsion comprising at least 8% weight of a volatile deactivant, wherein the deactivant is selected from:
a mint oil;
bois de rose oil;
oil of jasmine;
frankincense;
oil of bergamot;
oil of lemon grass;
or a component thereof.
